(19) United States
(12) Patent Application Publication
(10) Pub. No.: US 2008/0009841 A1
(43) Pub. Date: Jan. 10, 2008

(54) AUTO-CONTROL LASER SCANNING SYSTEM FOR DERMATOLOGY TREATMENT

(76) Inventor: Iku Kuo, Thaipao City (TW)

Correspondence Address: KAMRATH & ASSOCIATES P.A., 4825 OLSON MEMORIAL HIGHWAY, SUITE 245, GOLDEN VALLEY, MN, 55422, US

(21) Appl. No.: 11/428,727

(22) Filed: Jul. 5, 2006

(51) Int. Cl. A61B 18/18

(52) U.S. Cl. 606/9

(57) ABSTRACT

This invention is an auto control laser scanning system for dermatology treatment. This system would get the designated therapeutic area firstly by the photography device, and then transfer the image to the driver device. Additionally, choosing the range of the designated curing area by either manually or automatically, and after calculating the choosing area, it'll cut apart the area into many therapeutic spots (the actual number depends on the laser spot size.); in the meantime, the system also get the coordinate of every therapeutic spot. In accordance with the coordinate, this brand-new method would conduct laser to every designated therapeutic spot and start the treatment process. The operator does not need to operate manually at all, and consequently, the flaw of manual operation could be avoided, and the therapeutic area can also be controlled precisely. Moreover, by utilizing the image-orientated control system, the energy of laser will be distributed over skin steady and evenly. The other advantage of the device is avoiding eye contact during treatment procedure and prevents laser exposure to operator's eyes.

### FIELD OF THE INVENTION

By utilizing the camera and shooting the designated therapeutic area, the computer could calculate and analyze every coordinate of the therapeutic spot, and finally the system would make laser start the treatment procedure by every calculated coordinate automatically.

### BACKGROUND OF THE INVENTION

Laser has been applied on dermatology for decades, and also been known as a fruit of high-tech, but it still frightens most people. It's mainly because the lack of cognition of laser, especially the principle of laser treatment.

In fact, laser is one kinds of light energy; by lighting the skin surface, skin itself would affect four functions which are reflection, scattering, penetration, and absorption. By using specific wavelength, laser would reach the designated depth of tissue. Then the thermal energy, which is absorbed and also transformed by cell, would destroy and activate the tissue to reborn and rebuild the organization. In this way, some level of the therapy could be reached. The theory of laser skin therapy is that, some parts of the skin structure could absorb the stable wavelength of laser and produce high-energy laser in the twinkling of an eye, so the designated treatment result could be attained due to the destruction of one specific element in the skin. The laser lighting time is quite short, thus, the energy will not be spread to the organization around, and we can protect other harmless skin tissues could be protected from destruction.

Therefore, based on the destroy-designated area and choosing proper laser type and proper energy, the operator could destroy the object at will and without damaging other harmless or healthy tissues. That is to say, treating with laser is quite sophisticated and safe under such a prerequisite.

Dermatologists doubtless have all been trained professionally, but the point is, current laser skin therapeutic devices all need to be operated manually, and the steps of manual operation are as follows. First, the designated treatment area and its square measure are both decided by doctors' naked eyes; second, reaching the designated treatment area by holding laser projecting head by hand, and finally projecting laser by manual operation and starting the treating process. Base on these steps, we can easily find that the continuously long time surgery would probably cause some inaccuracies on the surgery and some side effects on doctors; for example, torpor of hand movement, improper control of muscle, and fatigue of eyes. And these flaws would do some unexpected harm on those healthy tissues and cells around. Moreover, re-treat at the same area due to the unavoidable human forgetfulness, or miscalculate laser projecting times, both would affect the treating result.

### SUMMARY OF THE INVENTION

For improving the discussed failure, this invention mainly uses the technology which photographs the designated treatment area through camera, and then delivers the image to the analyzing device. Additionally, choosing the range of the designated therapeutic area by either manually or automatically, then after calculating the choosing area, it'll cut apart the area into many therapeutic spots (it depends on the beam size of laser spot.); in the meantime, the system also get the coordinate of every therapeutic spot. In accordance with the coordinate, this inventing method will conduct the laser to every designated therapeutic spot and start the treatment procedure.

Hence, through our coordinate-oriented technology, the position of laser projection is conducted automatically and precisely. Thus, during the laser projecting therapy, the energy can be spread steadily and averagely. And the shortcomings of manual operation are avoidable since manual operation is no more needed.

This treatment can help the operator not only get the image through the camera, but also help the operator decide and choose the treating area conveniently. And during the treating procedure, the operator can protect eyes from contacting laser since they have no need to look directly at the treating area.

This invention can be fully understood and practiced through the following practicing examples tied in with attached figures.

### DETAILED DESCRIPTION OF THE EMBODIMENTS

Please compare the description with FIG. 1 and FIG. 2. This inventive system is composed of photography device A, laser conductive device, and analyzing driver device.

Photography device A: it could be either a video camera or a digital camera which could photograph along the designated projecting area.

Laser conductive device: this device is composed of a laser projecting head, scanning units both c1 and c2, and a reflecting unit b. By scanning unit c1 and c2, reflecting unit b could guide and project laser with different angles or positions.

Analyzing compound driving device: this system is composed of two computers and two drivers. First, one computer is used of getting the image from photography device A, and making the image show on the screen, meanwhile, carrying out the operation function of the image. Second, the other computer is used of sending the calculated data from the first computer and transferring it into driving data. Finally, the drivers would drive the scanning unit c1 and c2 to move by the directions.

Now, the whole therapeutic process is re-described completely below. First, photography device would photograph the designated treating area and convey the image into one computer of the analyzing compound driving device. Second, after getting the image, pick and decide the range of the designated treating area by manually or automatically.

Third, the computer would calculate and cut apart the selected area into several therapeutic spots which are just as small as the faculae, simultaneously, also calculate out the coordinates of each therapeutic spot. (As FIG. 3 shows, each square stands for the size of faculae, and also is the therapeutic spot. The light grays represent chosen regions, while deep grays represent the range of projecting lasers.)

Finally, conveying the calculated data into the other computer and starting to transfer the data into coordinates.

Through this step, the computer would make the driver drives scanning unit c1 and c2, thus, c1 and c2 would follow the instruction and conduct the laser to do treatment on deep grays.

All in all, the whole course of treatment is pretty precise and totally safe, and the energy of laser is stable and even. The main reason of this sound result is that all steps are controlled stably and automatically by computer; for instance, choosing and calculating the treating area, and projecting laser on the treating spot. For the operator, there are also two benefits for them. As the first one, they could examine the designated treating area clearer and more convenient, and could also determine and choose the treating area safely. For the other one, they no longer have to look directly at the treating area during the treatment procedure; and that is to say, the harm of strong laser light is totally avoidable.

The described practicing method has already provided a concrete explanation on this new system; also, it could be totally understood by those who are familiar with this technology. Thus, we have to announce that, even someone has some slightly changes on this new invention, they all still belong to our invention category; and that is to say, our invention can't be offended and easily achieved.

The above-mentioned preferred embodiments of the present invention are not intended to limit the scope of the present invention. Modifications and changes may be made in various embodiments of the present invention without leaving the scope and spirit of the present invention. The scope of the present invention is limited only by the language of the following claims.

Other features and advantages of the invention will become apparent from the following detailed description, taken in conjunction with the accompanying drawings.

### DESCRIPTION OF THE DRAWINGS

FIG. 1 shows a diagram of a system configuration of the present invention.

FIG. 2 shows a flow chart of processing of the present invention.

FIG. 3 shows a diagram of a curing area of the present invention.

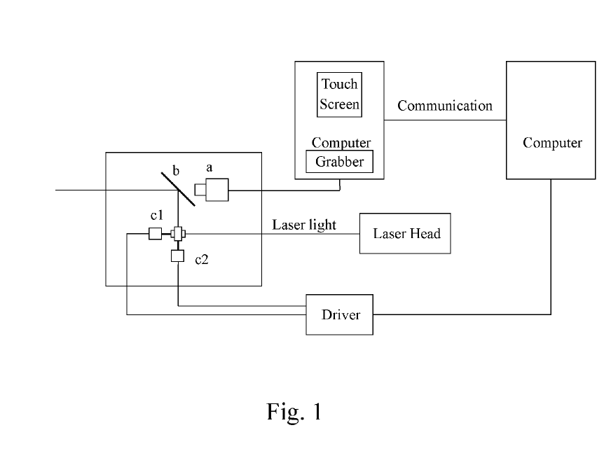

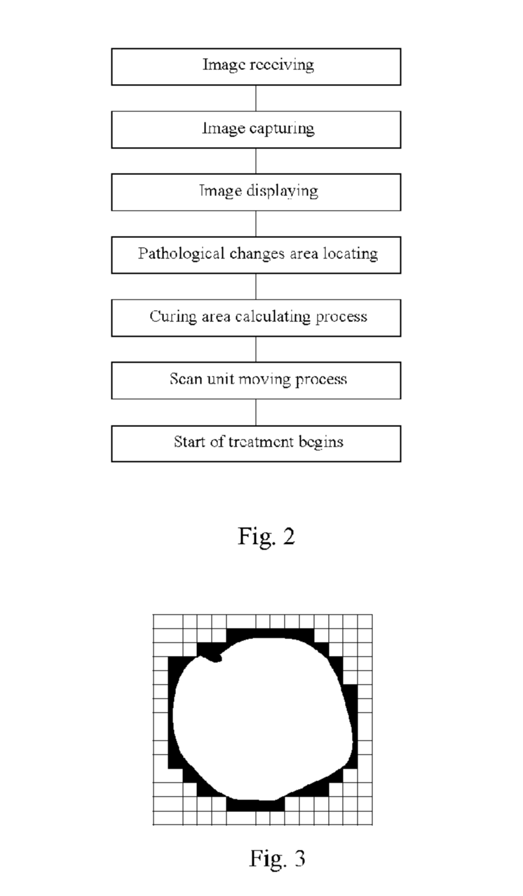

What is claimed is:

1. An auto-control laser scanning system for dermatology treatment, comprising:
a photography device, a laser conductive device and an analyzing driver device;
an image of a designated treating area is taken by the photography device so as to convey into and capture by the analyzing drive system; a range of the treating area is then selected and after further calculation be divided into plural number of therapeutic spots as the size of faculae, each coordinate of the therapeutic spots is also located at the same time; after that, laser is guided to each of the therapeutic spot according to the coordinate by the laser conductive device for treatment.

2. The laser scanning system of claim 1, wherein the photography device can be either video camera or digital camera.

3. The laser scanning system of claim 1, wherein the range could be selected either by hand or automatic.

4. The laser scanning system of claim 1, wherein the analyzing driver device is provided with an image obtaining plus calculating computer and a computer of laser conducting driver.
